(19) United States
(12) Patent Application Publication
(10) Pub. No.: US 2002/0082477 A1
(43) Pub. Date: Jun. 27, 2002

(54) Disposable laryngoscope handle protector

(76) Inventor: Yong Jin Kim, Homewood, IL (US)

Correspondence Address: McDONNELL BOEHNEN HULBERT & BERGHOFF, 300 South Wacker Drive, Chicago, IL, 60606, US

(21) Appl. No.: 09/742,656

(22) Filed: Dec. 21, 2000

(51) Int. Cl. A61B 1/267

(52) U.S. Cl. 600/186; 600/122

(57) ABSTRACT

A disposable protective sleeve used for covering laryngoscope handles that is integrally formed of a flexible material. The sheath defines a hollow cavity adapted to enclose a laryngoscope handle. After use, the sleeve is disposed of and the laryngoscope handle is covered with a new sleeve before being used again.

### FIELD OF INVENTION

The present invention relates, in general, to a disposable protective sleeve for a laryngoscope handle.

### BACKGROUND OF INVENTION

A laryngoscope is a hand held medical instrument used for visual examination of the larynx and trachea of a patient. It is also used in endotracheal intubation procedures to facilitate the insertion of an endotracheal tube through the glottis and into the trachea for general anaesthesia during surgery. While the instrument appears in two basic forms, an indirect laryngoscope and a direct laryngoscope, this application is concerned primarily with the direct-type instrument.

The direct laryngoscope is equipped with a built in illuminating device and a blade which moves the epiglottis and tongue forward to provide an unobstructed view of the larynx and the trachea. This instrument typically consists of a handle, a blade, and a connector fitting which holds the two together. The fitting is designed to permit quick attachment and detachment between the blade and the handle. A battery or batteries within the handle serve to power a light source. The latter may be a small incandescent lamp situated in the connector fitting or the upper portion of the handle. The lamp may be associated with a fiber optic light conductor extending along the blade to a point located rearwardly of the tip portion of the blade. In certain older instruments, the incandescent lamp is mounted on the blade slightly to the rear of the tip portion and connected by wire to the battery in the handle.

Potentially infectious agents such as blood or saliva is often transferred from the blade to a portion of the handle that the blade rests upon following the use of the laryngoscope. Under current medical practices, laryngoscope handles should be sterilized after each usage to avoid the transmission of infection from one patient to another. Unfortunately, this precaution is not always followed with adequate thoroughness. Due to the construction of these instruments, particularly deeply knurled surfaces of the handle to allow for a better grip for the physician using the laryngoscope, there remains a residual risk of the presence of bacterial or viral infection sources in those surfaces and resulting transmission of infection from patient to patient.

Several disposable coverings for laryngoscopes or portions thereof have been described in the prior art. Many of these prior art disposable covers cover the blade of the laryngoscope only, risking exposure of the patient to possible contamination from the connector fittings and/or the handle of the laryngoscope due to inadequate sterilization of these portions of the laryngoscope. Previous attempts to cover the portion of the laryngoscope handle which may come into contact with the patient have also been described. However, the covers tend to be shapeless “baggy” covers which do not easily conform to the contoured shape of the laryngoscope blade. Complicated vacuum evacuation systems have been suggested to conform such universal covers to the shape of the blade. Another approach which has been suggested is a condom-like elastic latex protecting barrier which consists of a series of concentric contiguous elastic portions with increasing diameters which may be placed at the tip of the laryngoscope blade and rolled over the blade and the entire handle. Although such a cover does provide protection for both the blade and handle of the laryngoscope, such elastic covers can fit so tightly to a laryngoscope that they are difficult to put on and remove.

Accordingly, there is a need in the medical field for a disposable sleeve that may be used on laryngoscope handles with varying shapes and sizes and protects the patient from indirect patient-to-patient transmission of infectious agents.

### SUMMARY OF INVENTION

The disposable protective sleeve of the present invention is easily placed onto a laryngoscope handle and provides complete coverage of those portions of the laryngoscope handle likely to come in contact with a patient. The protective sleeve comprises a flexible plastic sheath which preferably covers all of the handle. The sheath is preferably molded from a low cost, flexible, fluid-impervious plastic material, such as by injection molding or, preferably, by blow molding. The protective sleeve thus formed is economically disposable after each use.

The protective sleeve of the invention in a preferred form includes a contoured, fluid-impervious preformed sheath defining a hollow cavity, having an open end and a closed end, the open end having an aperture to receive a laryngoscope handle. The sheath covers the laryngoscope handle, particularly the portions that may come into contact with a contaminated laryngoscope blade. The preformed sheath has a semi-rigid shape which allows it to closely conform to the shape of the particular laryngoscope handle with which it is used. Generally, the sheath will cover at least the front portion of the handle that faces the blade and the patient when in use. Preferably, the open end of the sleeve is slightly greater than the closed end to allow the physician to readily insert and remove the handle from the sleeve.

The sleeve also preferably includes a means for securing the sleeve on the laryngoscope handle during use. For example, the shield may additionally include a first flap which can be wrapped around the laryngoscope handle and secured by a fastening means. The flap and fastening means serve to securely hold the disposable sleeve on the laryngoscope, preventing the disposable sleeve from accidentally slipping off the laryngoscope. The fastening means may comprise at least one peg integrally formed with and located on the sleeve and at least one hole in the flap, each hole of sufficient size to firmly encircle and hold each peg, such that when the flap is wrapped around the sleeve, at least one peg can be inserted into and firmly held in a hole.

As an alternative for holding the sleeve on the laryngoscope handle, the protective sleeve can feature one or more elastic bands that partially or wholly encircle the sheath to allow the sheath to grip onto the handle. The bands may be integrally formed with and located on the sleeve and serve to securely hold or grip the sleeve onto the handle. The bands may also comprise separate removable elastic concentric bands such as rubber or latex bands that firmly grip and fasten the sleeve onto the laryngoscope handle during use.

The sleeve also preferably includes means for removing the sleeve from the larynogoscope handle. For example, the sleeve may additionally feature a ziplock, a zipper, a tearoff strip, or a peel back strip along its longitudinal length to allow the user to quickly pull or tear the sleeve away from the handle.

The sheath portion of the present invention is preferably manufactured to accommodate the various lengths and sizes of laryngoscope handles known in the art. The sheath is preferably made from a material that gives it a semi-rigid shape that allows the sheath to be easily slid over the handle with minimal friction and without external lubrication, facilitating easy attachment of the sleeve to and removal of the sleeve from the laryngoscope handle without requiring that the sheath be rolled and/or stretched to draw it onto the handle. This material is also sufficiently flexible such that the sheath will distort as necessary to conform to the shape of the laryngoscope handle.

Further objects, features and advantages of the invention will be apparent from the following detailed description when taken in conjunction with the accompanying drawings.

### BRIEF DESCRIPTION OF THE DRAWINGS

FIG. 1 is a side elevational view of a common form of direct laryngoscope widely used for larynx and tracheal examination and to facilitate insertion of an endotracheal tube.

FIG. 2(a) is a side view of the disposable protective sleeve 15 prior to surrounding the laryngoscope handle during its use in examination of a patient. This embodiment shows an optional tearoff tab system 22 for facilitating the removal of the sleeve from the laryngoscope handle. FIG. 2(b) is a side view of the disposable protective sleeve with a funnel-shaped open end.

FIG. 3 is a side view of the disposable protective sleeve 15 also prior to surrounding the laryngoscope handle during its use in examination of a patient. This embodiment shows an optional elastic circular band for securing the sleeve to the handle during use.

FIG. 4 is an enlarged fragmentary view of (a) the sleeve illustrating the manner in which the sleeve is secured on the handle during use and (b) fastener peg with a flaired tip.

FIG. 5 is an enlarged fragmentary view illustrating the manner in which the sleeve is secured on the handle using a Velcro® fastener.

### DETAILED DESCRIPTION OF THE INVENTION

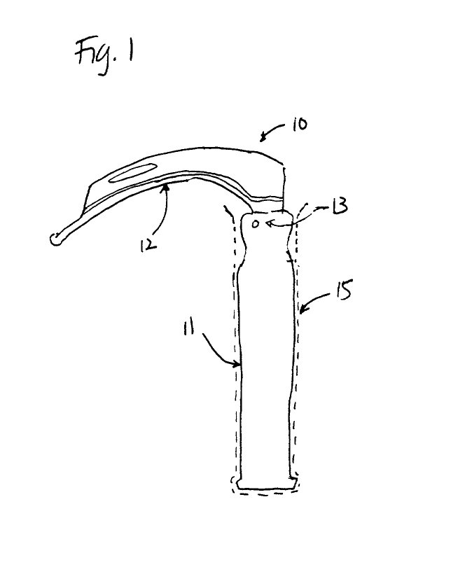

Referring more specifically to the drawing, the invention is there exemplified in connection with a typical direct laryngoscope 10 having a generally L-shaped configuration (FIG. 1). The instrument 10 comprises a handle 11, a somewhat arcuate shaped blade 12, and a connector fitting 13. The latter may be of the rigid interlocking type, or it may provide for angular adjustment of the blade relative to the handle. In the case of either construction, the instrument utilizes interfitting recesses, tongues and some type of locking mechanism (not detailed). The blade 12 has an inner surface which is curved laterally and acts as a guide surface which the physician can view into the patient's throat and which can be used to guide an intubation tube. While a curved detachable blade is illustrated, other types of blades such as a straight design may be used. A small electric lamp 14 is typically mounted adjacent to one edge of the blade 12 to assist in viewing the larynx and throat. The handle is fitted with a disposable, impervious sleeve 15.

Turning next to the disposable, impervious, sleeve 15 mentioned above, it will be noted that the device in the present instance comprises a hollow tube of generally round cross section with a diameter and length sufficient to cover to the full length of the handle 11. The hollow tube includes an open end 16 to receive the handle and a closed end 17. The diameter and length of the sleeve is such that the sleeve tends stretch longitudinally and diametrically to conform substantially to the shape of the laryngoscope handle (FIGS. 1,2).

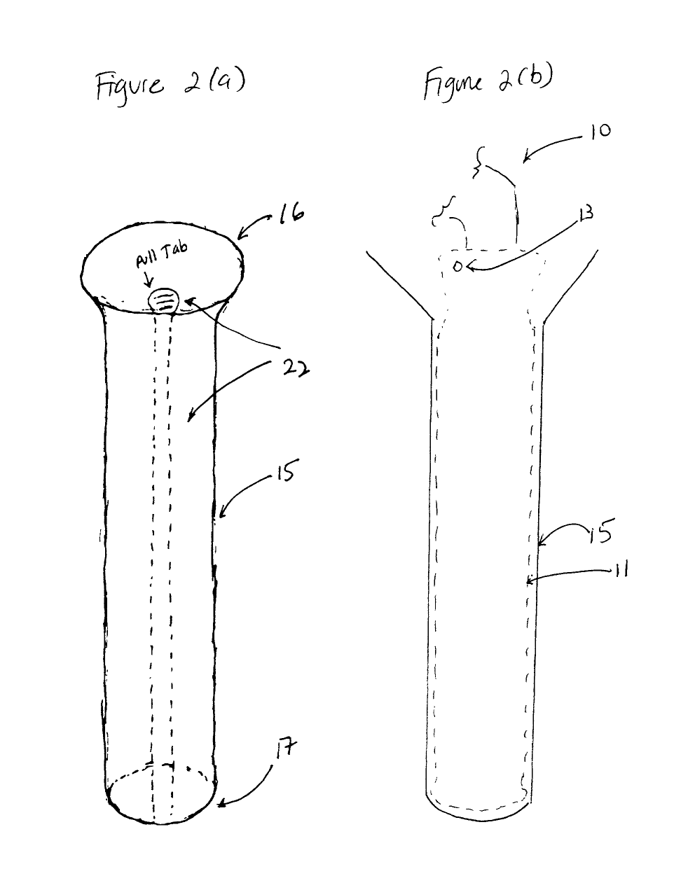

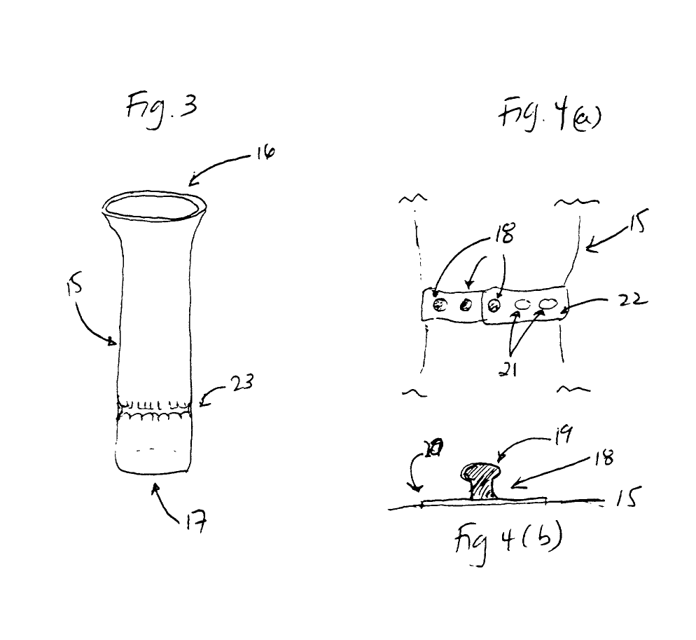

The disposable protective sleeve 15 for a laryngoscope handle in accordance with the present invention is also shown in FIGS. 1 and 2. The sleeve 15 has an closed end 17 and an open end 16. Preferably the open end of the tube is flaired (FIG. 2(a)) or funnel shaped (FIG. 2(b)) to facilitate the insertion of the handle 11 into the sleeve 15. Hence, the open end 16 has a round cross section and greater cross section area than the cross section area of closed end 17.

Sleeve 15 is preferably manufactured from a material such that the sleeve retains a semi-flexible shape which closely conforms to the handle 11 yet allows the physician to readily slide the handle into or from the sleeve. This form fitting shape allows the sheath to maintain close contact with the entire outside surface of the handle when it is in place. Form fitting preformed sleeves may be manufactured for each individual handle type and size, and such designs can be readily obtained using computer aided design techniques well known in the art. By molding the sleeve to closely conform to the style and size of handle on which the protective sleeve is to be used, several advantages are realized. The molded design allows the physician to easily slide the sleeve on or off the laryngoscope handle with a minimal amount of effort. In contrast, “condom” like protective sleeves which are elastically stretched over the handle are difficult to properly mount on the handle, and are also sometimes difficult to remove. Further, the preformed but close fitting design helps to minimize the volume occupied by the sleeve itself so that the sleeve does not interfere with the physician's ability to manipulate the laryngoscope.

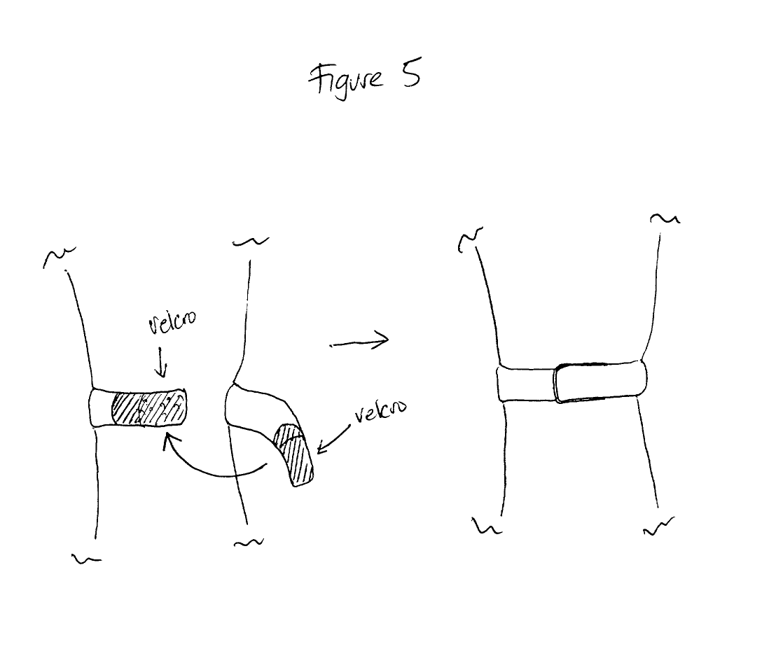

The protective sleeve 15 may additionally includes a means for holding the sleeve on the laryngoscope handle to avoid unintended removal or displacement of the protective sleeve during use. An example of structure which provides means for holding the sleeve 20 on the laryngoscope 21 is shown in FIG. 4. One or more pegs 18 are located on the sleeve 15. The pegs 18 may be integrally molded to the sleeve 15 from the same or different material as that of which the sleeve is formed. Alternatively, pegs 18 may be molded onto a flap 19 which is then attached to sleeve 15. The pegs 18 may have a flared tip 19 distal to the position of attachment of the pegs to the sleeve 15. At least one accompanying hole 21 is located on a flap 22 of the sleeve 15. The flap 22 can then be wrapped around the sleeve 15 containing handle 11 and the pegs 18 can be inserted through a hole or holes 21 to hold the sleeve in place, as shown in FIG. 4(a). By providing more than one hole 21 and more than one peg 18, the user can fasten the sleeve in place relatively tightly to handles 11 of various sizes. Alternate means for securing the sleeve onto the handle may also be used, and may consist of any suitable fastening means known in the art. Such means may consist of known fasteners such as Velcro® fasteners, zippers, chemical or adhesive sealants, and a linear or circular elastic band, wire, plastic, rubber or other similar material which may be wrapped around or twisted around the sleeve to hold the sleeve in place, and any other fastening means providing appropriate holding characteristics. The mechanisms described herein preferably allow easy fastening of the sleeve to and removal of the sleeve 15 from the laryngoscope handle 11. In practicing this invention, Velcro® fasteners are particularly preferred. During use, these fasteners generally lie flat and do not interfere with the physician's comfort during handling. FIG. 5 illustrates the use of a Velcro® fastener to secure the sleeve onto the handle.

The protective sleeve is preferably made of a relatively flexible, somewhat elastic but shape retaining, and fluid impervious synthetic polymer plastic that may be constructed such that the molded sheath fits closely to a particular style of handle and generally retains a shape which conforms to the shape of the handle prior to being placed on the blade. The sleeve may be formed integrally by injection molding or blow molding, or by other processes as desired, such as thermoforming, stretch forming, or by a solution process such as coating or dipping.

Suitable but non-limiting polymer materials that can be used for the disposable protective sleeve include polyolefins such as polyethylene or polypropylene or the products of homopolymerization or copolymerization of such olefins or diolefins as ethylene, propylene, butadiene, and isoprene. As typical examples, polyethylene, polypropylene, ethylene-vinyl acetate copolymer (EVA), polymer blends formed between EVA and various thermoplastic elastomers, and arbitrary combinations thereof may be cited. Besides, such polyesters as polyethylene terephthalate (PET), polybutylene terephthalate (PBT), and poly-1,4-cyclohexane dimethyl terephthalate (PCHT) and polyvinylidene chloride polyester materials. Other suitable materials include polyvinyl chloride, polyurethanes, polyamide materials such as nylon, polycarbonates, polysulfones, polyacrylates, and other similar materials. These materials are described in U.S. Pat. Nos. 6,132,844, 5,683,768, 5,523,004, 4,301,800, 4,222,379, and 4,112,989 which are incorporated by reference in their entirety herein. The protective sleeve may include a single layer or multiple bonded layers of polymer material of any suitable thickness sufficient to confer relatively flexible, somewhat elastic but shape retaining and fluid impervious properties. One particularly preferred polymer material is the type of materials that are commonly used in constructing IV bags for IV administration of fluids such as saline to patients. Optionally, the polymer material may have a surface texture to avoid or reduce the possibility of slippage during handling by the physician with wet surgical gloves.

The sleeve preferably includes means for removing the sleeve from the larynogoscope handle. For example, sleeve 15 may additionally feature a ziplock, a zipper, a tearoff strip, or a peel back strip along its longitudinal length to allow the user to quickly pull or tear the sleeve away from the handle. FIG. 1 shows a sleeve 15 including an optional tearoff strip system 22 for facilitating the removal of the sleeve 15 from a handle 11.

The material of the sleeve may be sterilizable, and the sterilized sleeve can be held in packaging where is kept in sterile condition until it is used if desired, although sterilization of the sleeve is not generally necessary since it typically will not be in contact with open wounds. The sleeve is preferably manufactured to dimensions slightly wider than the width of a particular laryngoscope handle to allow easy installation and removal from the handle. The material of the sheath may be selected in thickness in the general range from 0.001 inch to 0.120 inch. Preferably, the sheath material has a thickness of approximately 0.040 inch, and the shield portion has a thickness of approximately 0.060 inch, for materials such as LDPE or EVA.

The sleeve 15 need not necessarily precisely form-fit to the handle, as the material of the sleeve is preferably somewhat flexible such that, as the sheath is drawn over the handle the material of the sheath will bend and flex to conform more closely to handle. However, the material of the sleeve is also preferably rigid enough to retain generally the longitudinal shape of the handle prior to the installation of the sleeve on the handle for the benefits discussed above.

What is claimed is:

1. A disposable protective cover for covering a laryngoscope handle comprising a fluid-impervious preformed sleeve defining a hollow cavity to enclose therein portions of a laryngoscope handle, the sleeve having an open end and a closed end, the open end having an aperture through which the handle may be inserted into the cavity, the sleeve constructed to envelop the length of the handle and formed of a flexible plastic material molded to a preformed shape which conforms to the shape and contours of the handle while maintaining sufficient rigidity to retain the shape of the handle prior to installation of the sleeve on the handle.

2. The sleeve of claim 1 further including means for holding the sleeve on a laryngoscope handle.

3. The sleeve of claim 2 wherein the means for holding the sleeve on a laryngoscope handle comprises a flap on the sleeve and a fastening means for securing the flap such that when the sleeve is placed on a laryngoscope handle and the fastening means secured, the sleeve is firmly attached to the handle.

4. The sleeve of claim 3 wherein the means for holding the sleeve onto the laryngoscope handle comprises a Velcro® fastener.

5. The sleeve of claim 3 wherein the fastening means further comprising at least one peg located on the sleeve and the flap having at least one hole, each hole of sufficient size to firmly encircle and hold each peg, such that when the flap is wrapped around the sleeve enclosing the handle, at least one peg is engaged and firmly held in a hole, such that the sleeve is firmly attached to the laryngoscope handle.

6. The sleeve of claim 1 further including means for removing the sleeve from a laryngoscope handle.

7. The sleeve of claim 6 wherein the means for removing the sleeve from a laryngoscope handle comprise a ziplock, a zipper, a tearoff strip, or a peel back strip.

8. The sleeve of claim 1 wherein the sleeve is prepared from a material comprising low density polyethylene or ethylene vinyl acetate.
